(19) United States
(12) Patent Application Publication
(10) Pub. No.: US 2015/0057434 A1
(43) Pub. Date: Feb. 26, 2015

(54) METHODS OF PURIFYING HYDROPHOBIN

(71) Applicant: Michael Schelle, San Francisco, CA (US)

(72) Inventor: Michael Schelle, San Francisco, CA (US)

(73) Assignee: DANISCO US INC., Palo Alto, CA (US)

(21) Appl. No.: 14/112,215

(22) PCT Filed: Apr. 16, 2012
(86) PCT No.: PCT/US12/33728

§ 371 Date: Sep. 23, 2014

Related U.S. Application Data
(60) Provisional application No. 61/475,933, filed on Apr. 15, 2011.

(51) Int. Cl. C07K 1/30; C07K 14/37

(52) U.S. Cl. CPC C07K 1/30; C07K 14/37
USPC 530/350; 568/840

(57) ABSTRACT

The invention relates to a recovery and/or purification process of hydrophobins involving organic solvents and does not require separation techniques. In particular, the invention relates to a method for selective alcohol precipitation of hydrophobin II.

### RELATED APPLICATIONS AND INCORPORATION BY REFERENCE

This application claims priority to U.S. provisional patent application Ser. No. 61/475,933 filed Apr. 15, 2011. Reference is made to international patent application Serial No. PCT/US2009/046783 filed 9 Jun. 2009, which published as PCT Publication No. WO 2009/152176 on 17 Dec. 2009, Serial No. PCT/US2010/044964 filed 10 Aug. 2010, which published as PCT Publication No. WO 2011/019686 on 17 Feb. 2011, Serial No. PCT/US2010/044964 filed 10 Aug. 2010 and Serial No. PCT/US12/31104 filed 29 Mar. 2012.

The foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to a recovery and/or purification process of hydrophobins involving organic solvents and does not require separation techniques.

### BACKGROUND OF THE INVENTION

Hydrophobins are small proteins of about 100 to 150 amino acids which occur in filamentous fungi, for example Schizophyllum commune. They usually have 8 cysteine units. Hydrophobins can be isolated from natural sources, but can also be obtained by means of genetic engineering methods (see, e.g., WO 2006/082251 and WO 2006/131564).

Hydrophobins are spread in a water-insoluble form on the surface of various fungal structures, such as e.g. aerial hyphae, spores, fruiting bodies. The genes for hydrophobins could be isolated from ascomycetes, deuteromycetes and basidiomycetes. Some fungi have more than one hydrophobin gene, e.g. Schizophyllum commune, Coprinus cinereus, Aspergillus nidulans. Different hydrophobins are evidently involved in different stages of fungal development. The hydrophobins here are presumably responsible for different functions (van Wetter et al., 2000, Mol. Microbiol., 36, 201-210; Kershaw et al. 1998, Fungal Genet. Biol, 1998, 23, 18-33).

Hydrophobins identified to date are generally classed as either class I or class II. Both types have been identified in fungi as secreted proteins that self-assemble at interfaces into amphipathic films. Assemblages of class I hydrophobins are generally relatively insoluble whereas those of class II hydrophobins readily dissolve in a variety of solvents.

As biological function for hydrophobins, besides the reduction in the surface tension of water for the generation of aerial hyphae, the hydrophobicization of spores is also described (Wosten et al. 1999, Curr. Biol., 19, 1985-88; Bell et al. 1992, Genes Dev., 6, 2382-2394). Furthermore, hydrophobins serve to line gas channels in fruiting bodies of lichen and as components in the recognition system of plant surfaces by fungal pathogens (Lugones et al. 1999, Mycol. Res., 103, 635-640; Hamer & Talbot 1998, Curr. Opinion Microbiol., volume 1, 693-697).

Previously, hydrophobins were prepared only with moderate yield and purity using customary time-consuming protein-chemical purification (such as column purification and HPLC) and isolation methods (such as crystallization). Attempts of providing larger amounts of hydrophobins with the aid of genetic methods have also not been successful.

There is a need in the art for more effective method for faster and more economical methods for purifying large quantities of hydrophobin.

Citation or identification of any document in this application is not an admission that such document is available as prior art to the present invention.

### SUMMARY OF THE INVENTION

The present invention relates to a use or a method for purifying a biosurfactant, advantageously a hydrophobin, more advantageously hydrophobin II, which may comprise adding a precipitation agent, preferably an organic modifier, more preferably an alcohol, most preferably a C1-C3 alcohol, to a biosurfactant solution to generate a first precipitate, decanting a supernatant from the precipitation agent/biosurfactant solution and adding a same or different precipitation agent to the supernatant, to generate a second precipitate, wherein the second precipitate may be a purified biosurfactant, advantageously a purified hydrophobin, more advantageously purified hydrophobin II. The precipitation agent to generate the first precipitate may be the same or different precipitation agent to generate a second precipitate.

Preferably, the present invention relates to a use or a method for purifying hydrophobin II which may comprise adding a C1-C3 alcohol to a hydrophobin solution to generate a first precipitate, decanting a supernatant from the C1-C3 alcohol/hydrophobin solution and adding a C1-C3 alcohol to the supernatant, to generate a second precipitate, wherein the second precipitate may be purified hydrophobin II. The alcohol to generate the first precipitate may be the same or different alcohol to generate a second precipitate.

The invention, is based in part, on Applicant's surprising finding that pure class II hydrophobin can be purified by isopropanol precipitation from a crude concentrate.

In a first embodiment, the alcohol may be isopropanol. In an advantageous embodiment, about two to three volumes, preferably two to three volumes, more preferably two and a half volumes, of isopropanol may be added to generate the first precipitate. In another advantageous embodiment, about one volume, preferably one volume, of isopropanol may be added to the supernatant to generate the second precipitate.

In a second embodiment, the alcohol may be methanol. In an advantageous embodiment, about one to two volumes, preferably one to two volumes, more preferably one and a half volumes, of methanol may be added to generate the first precipitate. In another advantageous embodiment, about one volume, preferably one volume, of methanol may be added to the supernatant to generate the second precipitate.

In a third embodiment, the alcohol may be ethanol. In an advantageous embodiment, about one to two volumes, preferably one to two volumes, more preferably one and a half volumes, of ethanol may be added to generate the first precipitate. In another advantageous embodiment, about one volume, preferably one volume, of ethanol may be added to the supernatant to generate the second precipitate.

In the above embodiments, the first precipitate may be a brown precipitate and/or the second precipitate may be a white precipitate.

In a particularly advantageous embodiment, the use or method may be carried out at room temperature. Furthermore, the precipitation agent, preferably an organic modifier, more preferably an alcohol, most preferably a C1-C3 alcohol, may be recycled or reused for purifying a biosurfactant, advantageously a hydrophobin, more advantageously hydrophobin II.

The biosurfactant, advantageously a hydrophobin, more advantageously hydrophobin II, purified by the above use or method may be lyophilized. In particular, the purity of the purified biosurfactant, advantageously a hydrophobin, more advantageously hydrophobin II, may be assayed by SDS-PAGE, HPLC, mass spectrometry or amino acid analysis.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts purified HFBII analyzed by SDS-PAGE by diluting the samples in buffer as indicated (10 mM Tris-HCl, pH 8.0, 0.01% Tween-80) and mixing 2:1 with LDS Sample buffer containing 1× Reducing agent (Invitrogen). The samples were incubated at 90° C. for 5 min and 15 μL were loaded into each well of an SDS-PAGE gel (12%, 1 mM Bis-Tris, 10 lane, Invitrogen). The gel was run at 200 V for 35 min in 1×MES buffer (Invitrogen), stained using Coomassie Brilliant Blue, and destained (10% ethanol, 10% acetic acid). The resulting gel image shows a clear band for HFBII in the purified HFBII and no trace of the non-hydrophobin bands visible in the unpurified concentrate (1/100).

FIG. 2 depicts a RP-HPLC of a 1 mg/g solution of HFBII was prepared by diluting the sample in 10% acetonitrile. HFBII was separated by a reverse-phase HPLC system (Agilent) on a C5 column (Supelco Discovery C5, 300 Å, 5 μm, 2.1×100 mm) using a gradient of sodium phosphate buffer (“A”, 25 mM, pH 2.5) and acetonitrile (“B”, 0.05% TFA). The HFBII solution was injected (20 μL) onto the column (60° C.) and eluted by ramping from 10% solvent B to 70% B over 6 min at 0.8 mL/min. The system was returned to 10% B and equilibrated for 2 min before the next injection. HFBII was monitored by absorbance at 222 nm. HFBII elutes from the column at 4.38 min as one large peak and a small shoulder corresponding to the N-terminal phenylalanine truncation. No other peaks are observed in the chromatogram.

FIG. 3 depicts a mass spectrometry of purified HFBII (0.5 μL) that was spotted onto a stainless steel MALDI plate (Applied Biosystems), mixed with 0.5 μL of a saturated sinapinic acid solution (50% acetonitrile) and dried. The sample was analyzed by MALDI-TOF MS (Voyager, Applied Biosystems), acquiring in the positive mode between 4,000 and 20,000 m/z. The resulting spectrum shows a dominant peak at 7189.8 m/z, which corresponds to the mass of HFBII (calculated m+1=7189.4 m/z). The other peaks can be attributed to a known N-terminal phenylalanine truncation (m+1=7040.49 m/z) and the gas-phase HFBII dimer (14380 m/z).

### DETAILED DESCRIPTION OF THE INVENTION

As used herein, a “biosurfactant” or a “biologically produced surfactant” may be a protein, a glycolipid, a lipopeptide, a lipoprotein, a phospholipid, a neutral lipid or a fatty acid, and may decrease surface tension, such as the interfacial tension between water and a hydrophobic liquid, or between water and air, and that may be produced or obtained from a biological system. Biosurfactants include hydrophobins. Biosurfactants include lipopeptides and lipoproteins such as surfactin, peptide-lipid, serrawettin, viscosin, subtilisin, gramicidins, polymyxins. Biosurfactants include glycolipids such as rhamnolipids, sophorolipids, trehalolipids and cellobiolipids. Biosurfactants include polymers such as emulsan, biodispersan, mannan-lipid-protein, liposan, carbohydrate-protein-lipid, protein PA. Biosurfactants include particulates such as vesicles, fimbriae, and whole cells. Biosurfactants include glycosides such as saponins. Biosurfactants include fibrous proteins such as fibroin. The biosurfactant may occur naturally or it may be a mutagenized or genetically engineered variant not found in nature. This includes biosurfactant variants that have been engineered for lower solubility to help control foaming by lowering the biosurfactant solubility according to this invention. Biosurfactants include, but are not limited to, related biosurfactants, derivative biosurfactants, variant biosurfactants and homologous biosurfactants as described herein.

As used herein, a “biological system” comprises or is derived from a living organism such as a microbe, a plant, a fungus, an insect, a vertebrate or a life form created by synthetic biology. The living organism can be a variant not found in nature that is obtained by classical breeding, clone selection, mutagenesis and similar methods to create genetic diversity, or it can be a genetically engineered organism obtained by recombinant DNA technology. The living organism can be used in its entirety or it can be the source of components such as organ culture, plant cultivars, suspension cell cultures, adhering cell cultures or cell free preparations.

The biological system may or may not contain living cells when it sequesters the biosurfactant. The biological system may be found and collected from natural sources, it may be farmed, cultivated or it may be grown under industrial conditions. The biological system may synthesize the biosurfactant from precursors or nutrients supplied or it may enrich the biosurfactant from its environment.

As used herein, “production” relates to manufacturing methods for the production of chemicals and biological products, which includes, but is not limited to, harvest, collection, compaction, exsanguination, maceration, homogenization, mashing, brewing, fermentation, recovery, solid liquid separation, cell separation, centrifugation, filtration (such as vacuum filtration), formulation, storage or transportation.

As used herein, a “fermentation broth composition” refers to cell growth medium that contains a protein of interest, such as hydrophobin. The cell growth medium may include cells and/or cell debris, and may be concentrated. An exemplary fermentation broth composition is hydrophobin-containing, ultrafiltration-concentrated fermentation broth. Microfiltration is conventionally used to retain cell debris and pass proteins, e.g., for cell separation, while ultrafiltration is conventionally used to retain proteins and pass solutes, e.g., for concentration.

As used herein, the terms “polypeptide” and “protein” are used interchangeably to refer to polymers of any length comprising amino acid residues linked by peptide bonds. The conventional one-letter or three-letter code for amino acid residues is used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “culture solution” is a liquid comprising a biosurfactant and other soluble or insoluble components from which the biosurfactant of interest is intended to be recovered. Such components include other proteins, non-proteinaceous impurities such as cells or cell debris, nucleic acids, polysaccharides, lipids, chemicals such as antifoam, flocculants, salts, sugars, vitamins, growth factors, precipitants, and the like. A “culture solution” may also be referred to as “protein solution,” “liquid media,” “diafiltered broth,” “clarified broth,” “concentrate,” “conditioned medium,” “fermentation broth,” “lysed broth,” “lysate,” “cell broth,” or simply “broth.” The cells, if present, may be bacterial, fungal, plant, animal, human, insect, synthetic, etc.

As used herein, the term “recovery” refers to a process in which a liquid culture comprising a biosurfactant and one or more undesirable components is subjected to processes to separate the biosurfactant from at least some of the undesirable components, such as cells and cell debris, other proteins, amino acids, polysaccharides, sugars, polyols, inorganic or organic salts, acids and bases, and particulate materials.

As used herein, a “biosurfactant product” refers to a biosurfactant preparation suitable for providing to an end user, such as a customer. Biosurfactant products may include cells, cell debris, medium components, formulation excipients such as buffers, salts, preservative, reducing agents, sugars, polyols, surfactants, and the like, that are added or retained in order to prolong the functional shelf-life or facilitate the end use application of the biosurfactant. A biosurfactant product may also be purified.

As used herein, functionally and/or structurally similar biosurfactants are considered to be “related biosurfactants.” Such biosurfactants may be derived from organisms of different genera and/or species, or even different classes of organisms (e.g., bacteria and fungus). Related biosurfactants also encompass homologs determined by primary sequence analysis, determined by tertiary structure analysis, or determined by immunological cross-reactivity.

As used herein, the term “derivative biosurfactant” refers to a protein-based biosurfactant which is derived from a biosurfactant by addition of one or more amino acids to either or both the N- and C-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a biosurfactant derivative may be achieved by modifying a DNA sequence which encodes for the native protein, transformation of that DNA sequence into a suitable host, and expression of the modified DNA sequence to form the derivative protein. A “derivative biosurfactant” may also encompass biosurfactant derivatives where either lipid or carbohydrate moieties have been attached to protein backbone either during or after synthesis.

Related (and derivative) biosurfactants include “variant biosurfactant.” Variant protein-based biosurfactants differ from a reference/parent biosurfactant, e.g., a wild-type biosurfactant, by substitutions, deletions, and/or insertions at one or more amino acid residues. The number of differing amino acid residues may be one or more, for example, 1, 2, 3, 4, 5, 6, 7, 8, 9, 10, 15, 20, 30, 40, 50, or more amino acid residues. Variant biosurfactants share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a wildtype biosurfactant. A variant biosurfactant may also differ from a reference biosurfactant in selected motifs, domains, epitopes, conserved regions, and the like.

As used herein, “chimera” or “chimeric” refers to a single composition, advantageously a polypeptide, possessing multiple components, which may be from different organisms. As used herein, “chimeric” is used to refer to tandemly arranged moieties, including a biosurfactant or a variant biosurfactant thereof, that is engineered to result in a fusion protein possessing regions corresponding to the functions or activities of the individual protein moieties.

### One Embodiment

As used herein, the term “analogous sequence” refers to a sequence within a protein-based biosurfactant that provides similar function, tertiary structure, and/or conserved residues as the biosurfactant. For example, in epitope regions that contain an alpha-helix or a beta-sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some embodiments, the tertiary structure and/or conserved residues of the amino acids in the biosurfactant are located at or near the segment or fragment of interest. Thus, where the segment or fragment of interest contains, for example, an alpha-helix or a beta-sheet structure, the replacement amino acids preferably maintain that specific structure.

As used herein, the term “homologous biosurfactant” refers to a biosurfactant that has similar activity and/or structure to a reference biosurfactant. It is not intended that homologs necessarily be evolutionarily related. Thus, it is intended that the term encompass the same, similar, or corresponding biosurfactant(s) (i.e., in terms of structure and function) obtained from different organisms. In some embodiments, it is desirable to identify a homolog that has a quaternary, tertiary and/or primary structure similar to the reference biosurfactant.

The degree of homology between sequences may be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-395).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-360). The method is similar to that described by Higgins and Sharp (Higgins and Sharp (1989) CABIOS 5:151-153). Useful PILEUP parameters including a default gap weight of 3.00, a default gap length weight of 0.10, and weighted end gaps. Another example of a useful algorithm is the BLAST algorithm, described by Altschul et al. (Altschul et al. (1990) J. Mol. Biol. 215:403-410; and Karlin et al. (1993) Proc. Natl. Acad. Sci. USA 90:5873-5787). One particularly useful BLAST program is the WU-BLAST-2 program (See, Altschul et al. (1996) Meth. Enzymol. 266:460-480). Parameters “W,” “T,” and “X” determine the sensitivity and speed of the alignment. The BLAST program uses as defaults a word-length (W) of 11, the BLOSUM62 scoring matrix (See, Henikoff and Henikoff (1989) Proc. Natl. Acad. Sci. USA 89:10915) alignments (B) of 50, expectation (E) of 10, M′5, N′-4, and a comparison of both strands. Advantageously, a BLAST program or a program running the BLAST algorithm is utilized to determine sequence homology or identity levels.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity may be determined using known programs such as BLAST, ALIGN, and CLUSTAL using standard parameters. (See e.g., Altschul, et al. (1990) J. Mol. Biol. 215:403-410; Henikoff et al. (1989) Proc. Natl. Acad. Sci. USA 89:10915; Karin et al. (1993) Proc. Natl. Acad. Sci USA 90:5873; and Higgins et al. (1988) Gene 73:237-244). Software for performing BLAST analyses is publicly available through the National Center for Biotechnology Information. Also, databases may be searched using FASTA (Pearson et al. (1988) Proc. Natl. Acad. Sci. USA 85:2444-2448). Advantageously, a BLAST program or a program running the BLAST algorithm is utilized to determine sequence homology or identity levels. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “wild-type” and “native” biosurfactants are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the biosurfactant, preparing genomic libraries from organisms expressing the protein, and screening the libraries for the gene of interest by hybridization to the probes. Positively hybridizing clones are then mapped and sequenced.

The methods of the present invention can be applied to the isolation of a biosurfactant from a culture solution. Advantageously, the biosurfactant is a soluble extracellular biosurfactant that is secreted by microorganisms.

A group of exemplary biosurfactants are the hydrophobins, a class of cysteine-rich polypeptides expressed by filamentous fungi. Hydrophobins are small (˜100 amino acids) polypeptides known for their ability to form a hydrophobic coating on the surface of objects, including cells and man-made materials. First discovered in Schizophyllum commune in 1991, hydrophobins have now been recognized in a number of filamentous fungi. Based on differences in hydropathy and other biophysical properties, hydrophobins are categorized as being class I or class II.

The expression of hydrophobin conventionally requires the addition of a large amount of one or more antifoaming agents (i.e., antifoam) during fermentation. Otherwise, the foam produced by hydrophobin polypeptides saturates breather filters, contaminates vents, causes pressure build-up, and reduces protein yield. As a result, crude concentrates of hydrophobin conventionally contain residual amounts of antifoam, as well as host cell contaminants, which are undesirable in a hydrophobin preparation, particularly when the hydrophobin is intended as a food additive.

Hydrophobin can reversibly exist in forms having an apparent molecular weight that is greater than its actual molecular weight, which make hydrophobin well suited for recovery using the present methods. Liquid or foam containing hydrophobin can be continuously or periodically harvested from a fermentor for protein recovery as described, or harvested in batch at the end of a fermentation operation.

As used herein, the term “hydrophobin” may refer to a polypeptide capable of self-assembly at a hydrophilic/hydrophobic interface, and having the general formula (I):

(Y₁)ₙ-B₁-(X₁)ₐ-B₂-(X₂)_{b}-B₃-(X₃)_{c}-B₄-(X₄)_{d}-B₅-(X₅)ₑ-B₆-(X₆)_{f}-B₇-(X₇)_{g}-B₈-(Y₂)ₘ (I)

wherein: m and n are independently 0 to 2000; B₁, B₂, B₃, B₄, B₅, B₆, B₇ and B₈ are each independently amino acids selected from Cys, Leu, Ala, Pro, Ser, Thr, Met or Gly, at least 6 of the residues B₁ through B₈ being Cys; X₁, X₂, X₃, X₄, X₅, X₆, X₇, Y₁ and Y₂ independently represent any amino acid; a is 1 to 50; b is 0 to 5; c is 1 to 100; d is 1 to 100; e is 1 to 50; f is 0 to 5; and g is 1 to 100.

In some embodiments, the hydrophobin has a sequence of between 40 and 120 amino acids in the hydrophobin core. In some embodiments, the hydrophobin has a sequence of between 45 and 100 amino acids in the hydrophobin core. In some embodiments, the hydrophobin has a sequence of between 50 and 90, preferably 50 to 75, or 55 to 65 amino acids in the hydrophobin core. The term “the hydrophobin core” means the sequence beginning with the residue B₁ and terminating with the residue B₈.

In the formula (I), at least 6, or at least 7, or all 8 of the residues B₁ through B₈ are Cys.

In the formula (I), in some embodiments m is suitably 0 to 500, or 0 to 200, or 0 to 100, or 0 to 20, or 0 to 10, or 0 to 5, or 0.

In the formula (I), in some embodiments n is suitably 0 to 500, or 0 to 200, or 0 to 100, or 0 to 20, or 0 to 10, or 0 to 3.

In the formula (I), in some embodiments, a is 3 to 25, or 5 to 15. In one embodiment, a is 5 to 9.

In the formula (I), in some embodiments, b is 0 to 2, or preferably 0.

In the formula (I), in some embodiments, c is 5 to 50, or 5 to 40. In some embodiments, c is 11 to 39.

In the formula (I), in some embodiments, d is 2 to 35, or 4 to 23. In some embodiments, d is 8 to 23.

In the formula (I), in some embodiments, e is 2 to 15, or 5 to 12. In some embodiments, e is 5 to 9.

In the formula (I), in some embodiments, f is 0 to 2, or 0.

In the formula (I), in some embodiments, g is 3 to 35, or 6 to 21. In one embodiment, g is 6 to 18.

In some embodiments, the hydrophobins used in the present invention may have the general formula (II):

(Y₁)ₙ-B₁-(X₁)ₐ-B₂-(X₂)_{b}-B₃-(X₃)_{c}-B₄-(X₄)_{d}-B₅-(X₅)ₑ-B₆-(X₆)_{f}-B₇-(X₇)_{g}-B₈-(Y₂)ₘ (II)

wherein: m and n are independently 0 to 20; B₁, B₂, B₃, B₄, B₅, B₆, B₇ and B₈ are each independently amino acids selected from Cys, Leu, Ala, Pro, Ser, Thr, Met or Gly, at least 7 of the residues B₁ through B₈ being Cys; a is 3 to 25; b is 0 to 2; c is 5 to 50; d is 2 to 35; e is 2 to 15; f is 0 to 2; and g is 3 to 35.

In the formula (II), at least 7, or all 8 of the residues B₁ through B₈ are Cys.

In some embodiments, the hydrophobins used in the present invention may have the general formula (III):

(Y₁)ₙB₁-(X₁)ₐ-B₂-B₃-(X₃)_{c}-B₄-(X₄)_{d}-B₅-(X₅)ₑ-B₆-B₇-(X₇)_{g}-B₈-(Y₂)ₘ (III)

wherein: m and n are independently 0 to 20; B₁, B₂, B₃, B₄, B₅, B₆, B₇ and B₈ are each independently amino acids selected from Cys, Leu, Ala, Pro, Ser, Thr, Met or Gly, at least 7 of the residues B₁ through B₈ being Cys; a is 5 to 15; c is 5 to 40; d is 4 to 23; e is 5 to 12; and g is 6 to 21.

In the formula (III), at least 7, or 8 of the residues B₁ through B₈ are Cys.

In the formulae (I), (II) and (III), when 6 or 7 of the residues B₁ through B₈ are Cys, it is preferred that the residues B₃ through B₇ are Cys.

In the formulae (I), (II) and (III), when 7 of the residues B₁ through B₈ are Cys, in some embodiments: (a) B₁ and B₃ through B₈ are Cys and B₂ is other than Cys; (b) B₁ through B₇ are Cys and B₈ is other than Cys, (c) B₁ is other than Cys and B₂ through B₈ are Cys. When 7 of the residues B₁ through B₈ are Cys, it is preferred that the other residue is Ser, Pro or Leu. In some embodiments, B₁ and B₃ through B₈ are Cys and B₂ is Ser. In some embodiments, B₁ through B₇ are Cys and B₈ is Leu. In further embodiments, B₁ is Pro and B₂ through B₈ are Cys.

The cysteine residues of the hydrophobins used in the present invention may be present in reduced form or form disulfide (—S—S—) bridges with one another in any possible combination. In some embodiments, when all 8 of the residues B₁ through B₈ are Cys, disulfide bridges may be formed between one or more (preferably at least 2, more preferably at least 3, most preferably all 4) of the following pairs of cysteine residues: B₁ and B₆; B₂ and B₅; B₃ and B₄; B₇ and B₈. In some embodiments, when all 8 of the residues B₁ through B₈ are Cys, disulfide bridges may be formed between one or more (at least 2, or at least 3, or all 4) of the following pairs of cysteine residues: B₁ and B₂; B₃ and B₄; B₅ and B₆; B₇ and B₈.

Examples of specific hydrophobins useful in the present invention include those described and exemplified in the following publications: Linder et al., FEMS Microbiology Rev. 2005, 29, 877-896; Kubicek et al., BMC Evolutionary Biology, 2008, 8, 4; Sunde et al., Micron, 2008, 39, 773-784; Wessels, Adv. Micr. Physiol. 1997, 38, 1-45; Wösten, Annu. Rev. Microbiol. 2001, 55, 625-646; Hektor and Scholtmeijer, Curr. Opin. Biotech. 2005, 16, 434-439; Szilvay et al., Biochemistry, 2007, 46, 2345-2354; Kisko et al. Langmuir, 2009, 25, 1612-1619; Blijdenstein, Soft Matter, 2010, 6, 1799-1808; Wösten et al., EMBO J. 1994, 13, 5848-5854; Hakanpää et al., J. Biol. Chem., 2004, 279, 534-539; Wang et al.; Protein Sci., 2004, 13, 810-821; De Vocht et al., Biophys. J. 1998, 74, 2059-2068; Askolin et al., Biomacromolecules 2006, 7, 1295-1301; Cox et al.; Langmuir, 2007, 23, 7995-8002; Linder et al., Biomacromolecules 2001, 2, 511-517; Kallio et al. J. Biol. Chem., 2007, 282, 28733-28739; Scholtmeijer et al., Appl. Microbiol. Biotechnol., 2001, 56, 1-8; Lumsdon et al., Colloids & Surfaces B: Biointerfaces, 2005, 44, 172-178; Palomo et al., Biomacromolecules 2003, 4, 204-210; Kirkland and Keyhani, J. Ind. Microbiol. Biotechnol., Jul. 17 2010 (e-publication); Stübner et al., Int. J. Food Microbiol., 30 Jun. 2010 (e-publication); Laaksonen et al. Langmuir, 2009, 25, 5185-5192; Kwan et al. J. Mol. Biol. 2008, 382, 708-720; Yu et al. Microbiology, 2008, 154, 1677-1685; Lahtinen et al. Protein Expr. Purif., 2008, 59, 18-24; Szilvay et al., FEBS Lett., 2007, 5811, 2721-2726; Hakanpää et al., Acta Crystallogr. D. Biol. Crystallogr. 2006, 62, 356-367; Scholtmeijer et al., Appl. Environ. Microbiol., 2002, 68, 1367-1373; Yang et al, BMC Bioinformatics, 2006, 7 Supp. 4, S16; WO 01/57066; WO 01/57528; WO 2006/082253; WO 2006/103225; WO 2006/103230; WO 2007/014897; WO 2007/087967; WO 2007/087968; WO 2007/030966; WO 2008/019965; WO 2008/107439; WO 2008/110456; WO 2008/116715; WO 2008/120310; WO 2009/050000; US 2006/0228484; and EP 2042156A; the contents of which are incorporated herein by reference.

The hydrophobin can be any class I or class II hydrophobin known in the art, for example, hydrophobin from an Agaricus spp. (e.g., Agaricus bisporus), an Agrocybe spp. (e.g., Agrocybe aegerita), an Ajellomyces spp., (e.g., Ajellomyces capsulatus, Ajellomyces dermatitidis), an Aspergillus spp. (e.g., Aspergillus arvii, Aspergillus brevipes, Aspergillus clavatus, Aspergillus duricaulis, Aspergillus ellipticus, Aspergillus flavus, Aspergillus fumigatus, Aspergillus fumisynnematus, Aspergillus lentulus, Aspergillus niger, Aspergillus unilateralis, Aspergillus viridinutans), a Beauveria spp. (e.g., Beauveria bassiana), a Claviceps spp. (e.g., Claviceps fusiformis), a Coccidioides spp., (e.g., Coccidioides posadasii), a Cochliobolus spp. (e.g., Cochliobolus heterostrophus), a Crinipellis spp. (e.g., Crinipellis perniciosa), a Cryphonectria spp. (e.g., Cryphonectria parasitica), a Davidiella spp. (e.g., Davidiella tassiana), a Dictyonema spp. (e.g., Dictyonema glabratum), an Emericella spp. (e.g., Emericella nidulans), a Flammulina spp. (e.g., Flammulina velutipes), a Fusarium spp. (e.g., Fusarium culmorum), a Gibberella spp. (e.g., Gibberella moniliformis), a Glomerella spp. (e.g., Glomerella graminicola), a Grifola spp. (e.g., Grifola frondosa), a Heterobasidion spp. (e.g., Heterobasidion annosum), a Hypocrea spp. (e.g., Hypocrea jecorina, Hypocrea lixii, Hypocrea virens), a Laccaria spp. (e.g., Laccaria bicolor), a Lentinula spp. (e.g., Lentinula edodes), a Magnaporthe spp. (e.g., Magnaporthe oryzae), a Marasmius spp. (e.g., Marasmius cladophyllus), a Moniliophthora spp. (e.g., Moniliophthora perniciosa), a Neosartorya spp. (e.g., Neosartorya aureola, Neosartorya fennelliae, Neosartorya fischeri, Neosartorya glabra, Neosartorya hiratsukae, Neosartorya nishimurae, Neosartorya otanii, Neosartorya pseudofischeri, Neosartorya quadricincta, Neosartorya spathulata, Neosartorya spinosa, Neosartorya stramenia, Neosartorya udagawae), a Neurospora spp. (e.g., Neurospora crassa, Neurospora discreta, Neurospora intermedia, Neurospora sitophila, Neurospora tetrasperma), a Ophiostoma spp. (e.g., Ophiostoma novo-ulmi, Ophiostoma quercus), a Paracoccidioides spp. (e.g., Paracoccidioides brasiliensis), a Passalora spp. (e.g., Passalora fulva), Paxillus filamentosus Paxillus involutus), a Penicillium spp. (e.g., Penicillium camemberti, Penicillium chrysogenum, Penicillium marneffei), a Phlebiopsis spp. (e.g., Phlebiopsis gigantea), a Pisolithus (e.g., Pisolithus tinctorius), a Pleurotus spp., (e.g., Pleurotus ostreatus), a Podospora spp. (e.g., Podospora anserina), a Postia spp. (e.g., Postia placenta), a Pyrenophora spp. (e.g., Pyrenophora tritici-repentis), a Schizophyllum spp. (e.g., Schizophyllum commune), a Talaromyces spp. (e.g., Talaromyces stipitatus), a Trichoderma spp. (e.g., Trichoderma asperellum, Trichoderma atroviride, Trichoderma viride, Trichoderma reesii [formerly Hypocrea jecorina]), a Tricholoma spp. (e.g., Tricholoma terreum), a Uncinocarpus spp. (e.g., Uncinocarpus reesii), a Verticillium spp. (e.g., Verticillium dahliae), a Xanthodactylon spp. (e.g., Xanthodactylon flammeum), a Xanthoria spp. (e.g., Xanthoria calcicola, Xanthoria capensis, Xanthoria ectaneoides, Xanthoria flammea, Xanthoria karrooensis, Xanthoria ligulata, Xanthoria parietina, Xanthoria turbinata), and the like. Hydrophobins are reviewed in, e.g., Sunde, M et al. (2008) Micron 39:773-84; Linder, M. et al. (2005) FEMS Microbiol Rev. 29:877-96; and Wösten, H. et al. (2001) Ann. Rev. Microbiol. 55:625-46.

In a particularly advantageous embodiment, the hydrophobin is from a Trichoderma spp. (e.g., Trichoderma asperellum, Trichoderma atroviride, Trichoderma viride, Trichoderma reesii [formerly Hypocrea jecorina]), advantageously Trichoderma reseei.

In the art, as described herein, hydrophobins are divided into Classes I and II. It is known in the art that hydrophobins of Classes I and II can be distinguished on a number of grounds, including solubility. As described herein, hydrophobins self-assemble at an interface (e.g., a water/air interface) into amphipathic interfacial films. The assembled amphipathic films of Class I hydrophobins are generally re-solubilized only in strong acids (typically those having a pKₐ of lower than 4, such as formic acid or trifluoroacetic acid), whereas those of Class II are soluble in a wider range of solvents.

In some embodiments, the hydrophobin is a Class II hydrophobin. In some embodiments, the hydrophobin is a Class I hydrophobin.

In some embodiments, the term “Class II hydrophobin” includes a hydrophobin having the above-described self-assembly property at a water/air interface, the assembled amphipathic films being capable of redissolving to a concentration of at least 0.1% (w/w) in an aqueous ethanol solution (60% v/v) at room temperature. In some embodiments, the term “Class I hydrophobin” includes a hydrophobin having the above-described self-assembly property but which does not have this specified redissolution property.

In some embodiments, the term “Class II hydrophobin” includes a hydrophobin having the above-described self-assembly property at a water/air interface and the assembled amphipathic films being capable of redissolving to a concentration of at least 0.1% (w/w) in an aqueous sodium dodecyl sulfate solution (2% w/w) at room temperature. In some embodiments, the term “Class I hydrophobin” includes a hydrophobin having the above-described self-assembly property but which does not have this specified redissolution property.

Hydrophobins of Classes I and II may also be distinguished by the hydrophobicity/hydrophilicity of a number of regions of the hydrophobin protein.

In some embodiments, the term “Class II hydrophobin” includes a hydrophobin having the above-described self-assembly property and in which the region between the residues B₃ and B₄, i.e. the moiety (X₃)_{c}, is predominantly hydrophobic. In some embodiments, the term “Class I hydrophobin” includes a hydrophobin having the above-described self-assembly property but in which the region between the residues B₃ and B₄, i.e. the group (X₃)_{c}, is predominantly hydrophilic.

In some embodiments, the term “Class II hydrophobin” includes a hydrophobin having the above-described self-assembly property and in which the region between the residues B₇ and B₈, i.e. the moiety (X₇)_{g}, is predominantly hydrophobic. In some embodiments, the term “Class I hydrophobin” includes a hydrophobin having the above-described self-assembly property but in which the region between the residues B₇ and B₈, i.e. the moiety (X₇)_{g}, is predominantly hydrophilic.

In some embodiments, the term “Class II hydrophobin” includes a hydrophobin having the above-described self-assembly property and in which the region between the residues B₃ and B₄, i.e. the moiety (X₃)_{c}, is predominantly hydrophobic. In some embodiments, the term “Class I hydrophobin” includes a hydrophobin having the above-described self-assembly property but in which the region between the residues B₃ and B₄, i.e. the group (X₃)_{c}, is predominantly hydrophilic.

In some embodiments, the term “Class II hydrophobin” includes a hydrophobin having the above-described self-assembly property and in which the region between the residues B₇ and B₈, i.e. the moiety (X₇)_{g}, is predominantly hydrophobic. In some embodiments, the term “Class I hydrophobin” includes a hydrophobin having the above-described self-assembly property but in which the region between the residues B₇ and B₈, i.e. the moiety (X₇)_{g}, is predominantly hydrophilic.

The relative hydrophobicity/hydrophilicity of the various regions of the hydrophobin protein can be established by comparing the hydropathy pattern of the hydrophobin using the method set out in Kyte and Doolittle, J. Mol. Biol., 1982, 157, 105-132. A computer program can be used to progressively evaluate the hydrophilicity and hydrophobicity of a protein along its amino acid sequence. For this purpose, the method uses a hydropathy scale (based on a number of experimental observations derived from the literature) comparing the hydrophilic and hydrophobic properties of each of the 20 amino acid side-chains. The program uses a moving-segment approach that continuously determines the average hydropathy within a segment of predetermined length as it advances through the sequence. The consecutive scores are plotted from the amino to the carboxy terminus. At the same time, a midpoint line is printed that corresponds to the grand average of the hydropathy of the amino acid compositions found in most of the sequenced proteins. The method is further described for hydrophobins in Wessels, Adv. Microbial Physiol. 1997, 38, 1-45.

Class II hydrophobins may also be characterized by their conserved sequences.

In one embodiment, the Class II hydrophobins used in the present invention may have the general formula (IV):

(Y₁)ₙ-B₁-(X₁)ₐ-B₂-B₃-(X₃)_{c}-B₄-(X₄)_{d}-B₅-(X₅)ₑ-B₆-B₇-(X₇)_{g}-B₈-(Y₂)ₘ (IV)

wherein: m and n are independently 0 to 200; B₁, B₂, B₃, B₄, B₅, B₆, B₇ and B₈ are each independently amino acids selected from Cys, Leu, Ala, Ser, Thr, Met or Gly, at least 6 of the residues B₁ through B₈ being Cys; a is 6 to 12; c is 8 to 16; d is 2 to 20; e is 4 to 12; and g is 5 to 15.

In the formula (IV), in some embodiments, a is 7 to 11.

In the formula (IV), in some embodiments, c is 10 to 12. In some embodiments, c is 11.

In the formula (IV), in some embodiments, d is 4 to 18. In some embodiments, d is 4 to 16.

In the formula (IV), in some embodiments, e is 6 to 10. In some embodiments, e is 9 or 10.

In the formula (IV), in some embodiments, g is 6 to 12. In some embodiments, g is 7 to 10.

In some embodiments, the Class II hydrophobins used in the present invention may have the general formula (V):

(Y₁)ₙ-B₁-(X₁)ₐ-B₂-B₃-(X₃)^{c}-B₄-(X₄)_{d}-B₅-(X₅)ₑ-B₆-B₇-(X₇)_{g}-B₈-(Y₂)ₘ (V)

wherein: m and n are independently 0 to 10; B₁, B₂, B₃, B₄, B₅, B₆, B₇ and B₈ are each independently amino acids selected from Cys, Leu or Ser, at least 7 of the residues B₁ through B₈ being Cys; a is 7 to 11; c is 11; d is 4 to 18; e is 6 to 10; and g is 7 to 10.

In the formulae (IV) and (V), in some embodiments, at least 7 of the residues B₁ through B₈ are Cys, or all 8 of the residues B₁ through B₈ are Cys.

In the formulae (IV) and (V), in some embodiments, when 7 of the residues B₁ through B₈ are Cys, it is preferred that the residues B₃ through B₇ are Cys.

In the formulae (IV) and (V), in some embodiments, when 7 of the residues B₁ through B₈ are Cys, it is preferred that: (a) B₁ and B₃ through B₈ are Cys and B₂ is other than Cys; (b) B₁ through B₇ are Cys and B₈ is other than Cys, or (c) B₁ is other than Cys and B₂ through B₈ are Cys. In some embodiments, when 7 of the residues B₁ through B₈ are Cys, it is preferred that the other residue is Ser, Pro or Leu. In some embodiments, B₁ and B₃ through B₈ are Cys and B₂ is Ser. In some embodiments, B₁ through B₇ are Cys and B₈ is Leu. In some embodiments, B₁ is Pro and B₂ through B₈ are Cys.

In the formulae (IV) and (V), in some embodiments, the group (X₃)_{c} comprises the sequence motif ZZXZ, wherein Z is an aliphatic amino acid; and X is any amino acid. The term “aliphatic amino acid” means an amino acid selected from the group consisting of glycine (G), alanine (A), leucine (L), isoleucine (I), valine (V) and proline (P).

In some embodiments, the group (X₃)_{c} comprises the sequence motif selected from the group consisting of LLXV, ILXV, ILXL, VLXL and VLXV. In some embodiments, the group (X₃)_{c} comprises the sequence motif VLXV.

In the formulae (IV) and (V), in some embodiments, the group (X₃)_{c} comprises the sequence motif ZZXZZXZ, wherein Z is an aliphatic amino acid; and X is any amino acid. In some embodiments, the group (X₃)_{c} comprises the sequence motif VLZVZXL, wherein Z is an aliphatic amino acid; and X is any amino acid.

Applicants have observed that hydrophobin II produced by other methods can result in one or more amino acids clipped at the C terminus. The methods of the present invention will precipitate both full length hydrophobin II and hydrophobin II clipped at the C terminus.

Hydrophobin-like proteins (e.g.“chaplins”) have also been identified in filamentous bacteria, such as Actinomycete and Streptomyces sp. (WO01/74864; Talbot, 2003, Curr. Biol, 13: R696-R698). These bacterial proteins by contrast to fungal hydrophobins, may form only up to one disulfide bridge since they may have only two cysteine residues. Such proteins are an example of functional equivalents to hydrophobins, and another type of molecule within the ambit of biosurfactants of methods herein.

Fermentation to produce the biosurfactant is carried out by culturing the host cell or microorganism in a liquid fermentation medium within a bioreactor or fermenter. The composition of the medium (e.g. nutrients, carbon source etc.), temperature and pH are chosen to provide appropriate conditions for growth of the culture and/or production of the biosurfactant. Air or oxygen-enriched air is normally sparged into the medium to provide oxygen for respiration of the culture.

As used herein, a “fermentation broth composition” refers to cell growth medium that contains a protein of interest, such as hydrophobin. The cell growth medium may include cells and/or cell debris, and may be concentrated. An exemplary fermentation broth composition is hydrophobin-containing, ultrafiltration-concentrated fermentation broth. Microfiltration is conventionally used to retain cell debris and pass proteins, e.g., for cell separation, while ultrafiltration is conventionally used to retain proteins and pass solutes, e.g., for concentration.

Advantageously, a cross-flow membrane filtration recovery method may allow for a preparation of a hydrophobin concentration as described in PCT Patent Publication WO 2011/019686 which is incorporated by reference. In other embodiments, size exclusion filtration and crystallization may also allow for a preparation of a hydrophobin concentration.

The invention encompasses a method for purifying a biosurfactant, advantageously a hydrophobin, more advantageously hydrophobin II, which may comprise adding a precipitation agent to a biosurfactant, advantageously a hydrophobin, more advantageously hydrophobin II, solution to generate a first precipitate, decanting a supernatant from the precipitation agent/biosurfactant solution and adding the same or different precipitation agent to the supernatant, to generate a second precipitate, wherein the second precipitate may be purified biosurfactant, advantageously a purified hydrophobin, more advantageously purified hydrophobin II. Examples of suitable precipitation agents include, but are not limited to, inorganic salts, organic modifiers, and combinations thereof. The precipitation agent to generate the first precipitate may be the same or different than the precipitation agent to generate the second precipitate. Any combination of suitable precipitation agents may be contemplated by the present invention.

As used herein, organic modifiers are organic solvents that are miscible in water. One of skill in the art may ascertain as to whether a particular organic modifier is miscible in water using knowledge and/or methods known to those of ordinary skill in the chemical art. For example, the absence of a biphasic mixture when a particular organic modifier is added to water indicates that it is miscible in water. The presence of a biphasic mixture when a particular organic modifier is added to water indicates that it is immiscible in water. Examples of suitable organic modifiers include, but are not limited to, acetonitrile, acetone, alcohols, dimethyl formamide (DMF), dimethyl sulfoxide (DMSO), dioxane, and tetrahydrofuran (THF).

Advantageously, the precipitation agent is an alcohol, more advantageously a C1-C4 alcohol, most advantageously a C1-C3 alcohol. Alcohols may be monohydric or polyhydric. Examples of C1-C4 alcohols include, but are not limited to, methanol, ethanol, 1-propanol, 2-propanol (isopropanol or isopropyl alcohol), t-butanol, and the like. Examples of C1-C3 alcohols include, but are not limited to, methanol, ethanol, 1-propanol, and 2-propanol (isopropanol or isopropyl alcohol). Advantageously the alcohol is methanol, ethanol or isopropyl alcohol.

Without being bound by theory, the amount of precipitation agent, preferably an alcohol, more preferably a C1-C4 alcohol, most preferably a C1-C3 alcohol, added would primarily precipitate proteins in the first precipitate. After decanting the supernatant from the precipitation agent/biosurfactant solution and adding the same or different precipitation agent, preferably an alcohol, more preferably a C1-C4 alcohol, most preferably a C1-C3 alcohol, to the supernatant may generate a biosurfactant, advantageously a hydrophobin, more advantageously hydrophobin II in a second precipitate.

A biosurfactant, advantageously a hydrophobin, more advantageously hydrophobin II may be precipitated with isopropyl alcohol or isopropanol. About two to three volumes of isopropanol, advantageously about two and a half volumes of isopropanol, may be added to one volume of biosurfactant, advantageously a hydrophobin, more advantageously hydrophobin II, solution in water to generate a first precipitate, which advantageously may be a brown precipitate. The supernatant may be decanted and a biosurfactant, advantageously a hydrophobin, more advantageously hydrophobin II, may be precipitated as a second precipitate, which advantageously may be a white precipitate, by adding about one volume of isopropanol.

Advantageously, the isopropanol is added. Two to three volumes of isopropanol, advantageously two and a half volumes of isopropanol, may be added to one volume of biosurfactant, advantageously a hydrophobin, more advantageously hydrophobin II, solution in water. The initial precipitate may form after about 15 minutes in a stirred solution, although one of skill in the art may ascertain the formation of an initial precipitate (which may be a brown precipitate). The second precipitation of biosurfactant, advantageously a hydrophobin, more advantageously hydrophobin II, form after about 10 minutes in a stirred solution, although one of skill in the art may ascertain the formation of a second precipitate, which advantageously may be a white precipitate.

A biosurfactant, advantageously a hydrophobin, more advantageously hydrophobin II, may be precipitated with methanol. About one to two volumes of methanol, advantageously about one and a half volumes of methanol, may be added to one volume of biosurfactant, advantageously a hydrophobin, more advantageously hydrophobin II, solution in water to generate a first precipitate, which advantageously may be a brown precipitate. The supernatant may be decanted and a biosurfactant, advantageously a hydrophobin, more advantageously hydrophobin II, may be precipitated as a second precipitate, which advantageously may be a white precipitate, by adding about three volumes of methanol.

Advantageously, the methanol is added at room temperature. One to two volumes of methanol, advantageously one and a half volumes of methanol, may be added to one volume of a biosurfactant, advantageously a hydrophobin, more advantageously hydrophobin II, solution in water. The initial precipitate may form after about 15 minutes in a stirred solution, although one of skill in the art may ascertain the formation of an initial precipitate, which advantageously may be a brown precipitate. The second precipitation of biosurfactant, advantageously a hydrophobin, more advantageously hydrophobin II forms after about 10 minutes in a stirred solution, although one of skill in the art may ascertain the formation of a second precipitate, which advantageously may be a white precipitate.

A biosurfactant, advantageously a hydrophobin, more advantageously hydrophobin II, may be precipitated with ethanol. About one to two volumes of ethanol, advantageously about one and a half volumes of ethanol, may be added to one volume of a biosurfactant, advantageously a hydrophobin, more advantageously hydrophobin II, solution in water to generate a first precipitate, which advantageously may be a brown precipitate. The supernatant may be decanted and biosurfactant, advantageously a hydrophobin, more advantageously hydrophobin II, may be precipitated as a second precipitate, which advantageously may be a white precipitate, by adding about three volumes of ethanol.

Advantageously, the ethanol is added at room temperature. One to two volumes of ethanol, advantageously one and a half volumes of ethanol, may be added to one volume of a biosurfactant, advantageously a hydrophobin, more advantageously hydrophobin II, solution in water. The initial precipitate may form after about 15 minutes in a stirred solution, although one of skill in the art may ascertain the formation of an initial precipitate, which advantageously may be a brown precipitate. The second precipitation of biosurfactant, advantageously a hydrophobin, more advantageously hydrophobin II, form after about 10 minutes in a stirred solution, although one of skill in the art may ascertain the formation of a second precipitate, which advantageously may be a white precipitate.

One of skill in the art may ascertain as to whether a particular a precipitation agent, preferably an organic modifier, more preferably an alcohol, as well as determine specific volumes of the particular precipitation agent by determining if an initial precipitate is present and further by determining if a biosurfactant, advantageously a hydrophobin, more advantageously hydrophobin II, precipitates from the supernatant, as determined by the presence of a second precipitate. Advantageously, the initial or first precipitate is brown and the second precipitate is white. Such observations are within the purview of a skilled artisan.

A particularly advantageous embodiment of the present invention is that the precipitation agent, preferably an organic modifier, more preferably an alcohol, may be reused or recycled, thereby reducing waste. In other words, the precipitation agent, preferably an organic modifier, more preferably an alcohol, used to precipitate the biosurfactant, advantageously a hydrophobin, more advantageously hydrophobin II, may be reused or recycled for additional precipitation of the biosurfactant, advantageously a hydrophobin, more advantageously hydrophobin II.

The precipitation may be harvested by centrifugation and lyophilized, which may result in a fine powder. In an advantageous embodiment, the powder is white. The powder may be dissolved in a solvent (such as water, a water/alcohol mix, a water/organic mix (such as water/acetonitrile), an organic solvent, such as DMSO or DMF) and may be frozen.

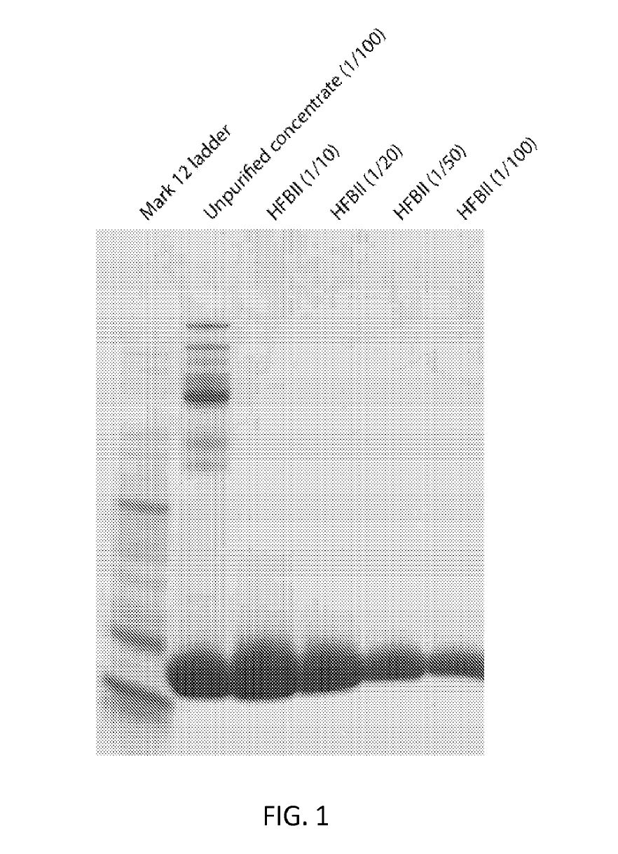

The purity of the biosurfactant, advantageously a hydrophobin, more advantageously hydrophobin II, may be assessed by any method known in the art, such as, but not limited to, SDS-PAGE, HPLC, mass spectrometry and amino acid analysis. For example, FIGS. 1-3 and Table 1 are illustrative of the purity of hydrophobin II as isolated by the herein disclosed methods.

Although the present invention and its advantages have been described in detail, it should be understood that various changes, substitutions and alterations can be made herein without departing from the spirit and scope of the invention as defined in the appended claims.

The present invention will be further illustrated in the following Example which is given for illustration purposes only and are not intended to limit the invention in any way.

### Example

### Isopropanol Precipitation of Hydrophobin II

Unpurified hydrophobin concentrate (200 mL, 150 mg/g) was added to a 1 L glass beaker and slowly mixed with 500 mL (2.5 volumes) isopropanol. The solution was stirred at room temperature for 15 min, resulting in the formation of a brown precipitate. The mixture was centrifuged (5 min, 10,000 rpm, Sorvall Untracentrifuge, SLA-1500 rotor) and the HFBII-containing supernatant was decanted into a clean 1 L glass beaker. Isopropanol (1 volume) was added to the supernatant and stirred for 10 min at room temperature, resulting in fine white precipitate. This precipitate was harvested by centrifugation (10 min, 10,000 rpm), transferred to a 1200 mL lyophilization jar and lyophilized for 62 hours, resulting in a fine white powder. The powder was dissolved in 100 mL deionized water and frozen.

Dry Solids Analysis.

The solids content of 1 g of purified HFBII was analyzed by microwave drying (Omnimark μWave Moisture Analyzer), resulting in 7.33% dry solids.

SDS-PAGE.

As shown in FIG. 1, purified HFBII was analyzed SDS-PAGE by diluting the samples in buffer as indicated (10 mM Tris-HCl, pH 8.0, 0.01% Tween-80) and mixing 2:1 with LDS Sample buffer containing 1× Reducing agent (Invitrogen). The samples were incubated at 90° C. for 5 min and 15 μL were loaded into each well of an SDS-PAGE gel (12%, 1 mM Bis-Tris, 10 lane, Invitrogen). The gel was run at 200 V for 35 min in 1×MES buffer (Invitrogen), stained using Coomassie Brilliant Blue, and destained (10% ethanol, 10% acetic acid). The resulting gel image shows a clear band for HFBII in the purified sample and no trace of the non-hydrophobin bands visible in the unpurified concentrate (1/100).

RP-HPLC.

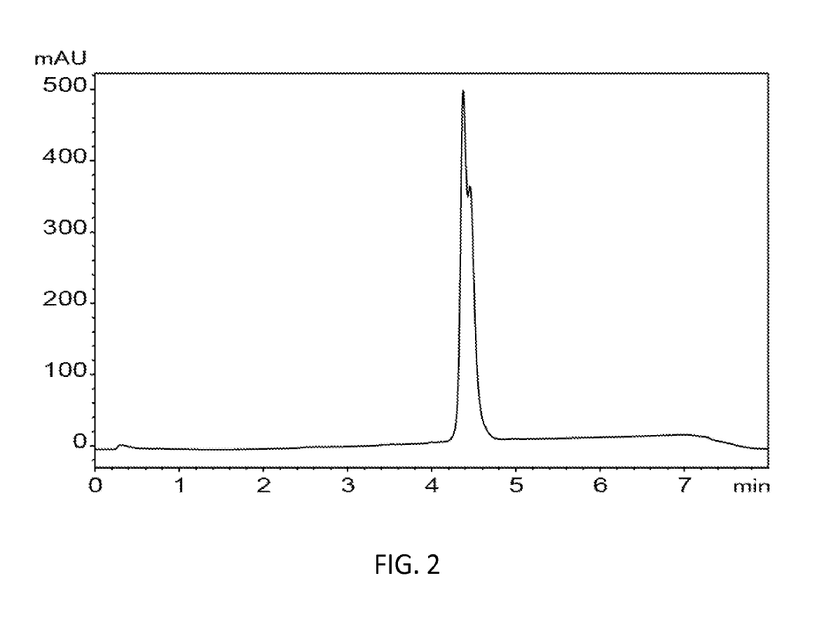

As shown in FIG. 2., a 1 mg/g solution of HFBII was prepared by diluting the sample in 10% acetonitrile. HFBII was separated by a reverse-phase HPLC system (Agilent) on a C5 column (Supelco Discovery C5, 300 Å, 5 μm, 2.1×100 mm) using a gradient of sodium phosphate buffer (“A”, 25 mM, pH 2.5) and acetonitrile (“B”, 0.05% TFA). The HFBII solution was injected (20 μL) onto the column (60° C.) and eluted by ramping from 10% solvent B to 70% B over 6 min at 0.8 mL/min. The system was returned to 10% B and equilibrated for 2 min before the next injection. HFBII was monitored by absorbance at 222 nm. HFBII elutes from the column at 4.38 min as one large peak and a small shoulder corresponding to the N-terminal phenylalanine truncation. No other peaks are observed in the chromatogram.

Mass Spectrometry.

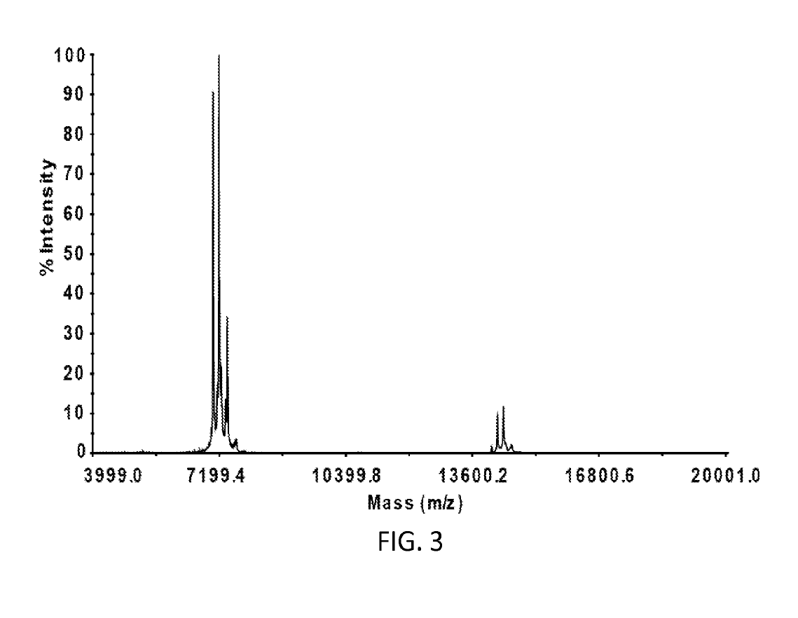

As shown in FIG. 3., Purified HFBII (0.5 μL) was spotted onto a stainless steel MALDI plate (Applied Biosystems), mixed with 0.5 μL of a saturated sinapinic acid solution (50% acetonitrile) and dried. The sample was analyzed by MALDI-TOF MS (Voyager, Applied Biosystems), acquiring in the positive mode between 4,000 and 20,000 m/z. The resulting spectrum shows a dominant peak at 7189.8 m/z, which corresponds to the mass of HFBII (calculated m+1=7189.4 m/z). The other peaks can be attributed to a known N-terminal phenylalanine truncation (m+1=7040.49 m/z) and the gas-phase HFBII dimer (14380 m/z).

Amino Acid Analysis.

Purified HFBII (1 mL) was analyzed for Amino Acid Analysis in duplicate by an outside laboratory (AAA Services, Inc.). As shown in Table 1, the results indicate that HFBII is present at 63.2 mg/g and is the dominant protein in solution as indicated by the similarity between the calculated and observed amino acid composition.

TABLE 1

| Amino | known | pMole | EXP | Int. | |
|---|---|---|---|---|---|
| Acid | Comp | Anal | Comp | Comp | uMoles/ml |
| CYSO2 | 0 | 0 | 0.00 | 0 | 0.0000 |
| HYP (Z) | 0 | 0 | 0.00 | 0 | 0.0000 |
| ASP (D) | 6 | 2695 | 6.14 | 6 | 13.4775 |
| THR (T) | 6 | 2569 | 6.14 | 6 | 12.8453 |
| SER (S) | 3 | 1213 | 3.04 | 3 | 6.0667 |
| GLU (E) | 3 | 1425 | 3.24 | 3 | 7.1239 |
| PRO (P) | 5 | 2145 | 4.88 | 5 | 10.7257 |
| GLY (G) | 5 | 2276 | 5.18 | 5 | 11.3791 |
| ALA (A) | 10 | 4355 | 9.91 | 10 | 21.7754 |
| VAL (V) | 6 | 2664 | 6.06 | 6 | 13.3181 |
| MET (M) | 0 | 0 | 0.00 | 0 | 0.0000 |
| ILE (I) | 4 | 1720 | 3.92 | 4 | 8.6007 |
| LEU (L) | 7 | 3132 | 7.13 | 7 | 15.6609 |
| NLE | 0 | 0 | 0.00 | 0 | 0.0000 |
| TYR (Y) | 0 | 0 | 0.00 | 0 | 0.0000 |
| PHE (F) | 3 | 1278 | 2.91 | 3 | 6.3908 |
| HIS (H) | 1 | 452 | 1.03 | 1 | 2.2578 |
| HLYS | 0 | 0 | 0.00 | 0 | 0.0000 |
| LYS (K) | 4 | 1742 | 3.97 | 4 | 8.7123 |
| ARG (R) | 0 | 0 | 0.00 | 0 | 0.0000 |
| Total AA's | 63 | | | | |
| Total pMole amino acid | | 27667 | | Calc. pMole protein 439 | |
| Total pMole hydrolyzed | | 1757 | | | |
| Conc pMol/ul | 8786 uM | | | | |
| Total ugrams | 12.6 | | | | |
| Conc. mg/ml | 63.2 | | | | |

Alcohol Precipitation Scan.

Alternative alcohols were assayed for their ability to selectively precipitate HFBII. Co-solvents were added to one volume HFBII concentrate, centrifuged at 14,000 rpm for 5 minutes and assessed for precipitation. Addition of one and two volumes of methanol resulted in a dark brown or light brown precipitate respectively. The supernatant of this solution was mixed with 3 more volumes of methanol (5 total), resulting in a large white precipitate exactly as observed with isopropanol. Similar results were observed with ethanol as the co-solvent. Glycerol was not able to precipitate any protein at a 4:1 ratio. Also, 1-butanol and 1-octanol did not precipitate any proteins and instead form a biphasic mixture. Thus, small-chain (C3 or less) alcohols are effective at selectively precipitating HFBII.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed:

1. A method for purifying hydrophobin II comprising adding a C₁-C₃ alcohol to a hydrophobin solution to generate a first precipitate, decanting a supernatant from the C₁-C₃ alcohol/hydrophobin solution and adding the C₁-C₃ alcohol to the supernatant, to generate a second precipitate, wherein the second precipitate comprises purified hydrophobin II.

2. The method of claim 1, wherein the C₁-C₃ alcohol is isopropanol.

3. The method of claim 2, wherein about two to three volumes of isopropanol is added to generate the first precipitate.

4. (canceled)

5. The method of claim 2, wherein about one volume of isopropanol is added to the supernatant to generate the second precipitate.

6. The method of claim 1, wherein the C₁-C₃ alcohol is methanol.

7. The method of claim 6, wherein about one to two volumes of methanol is added to generate the first precipitate.

8. (canceled)

9. The method of claim 6, wherein about three volume of methanol is added to the supernatant to generate the second precipitate.

10. The method of claim 1, wherein the C₁-C₃ alcohol is ethanol.

11. The method of claim 10, wherein about one to two volumes of ethanol is added to generate the first precipitate.

12. (canceled)

13. The method of claim 10, wherein about one volume of ethanol is added to the supernatant to generate the second precipitate.

14. The method of claim 1, wherein the method is performed at room temperature.

15. (canceled)

16. The method of claim 1, wherein the purified hydrophobin II is lyophilized.

17. The method of claim 1, wherein purity of the purified hydrophobin II is assayed by SDS-PAGE, HPLC, mass spectrometry or amino acid analysis.

18. Use of a C1-C3 alcohol to purify hydrophobin II comprising method of claim 1.

19-20. (canceled)

21. The method of claim 3, wherein about one volume of isopropanol is added to the supernatant to generate the second precipitate.

22. The method of claim 7, wherein about three volume of methanol is added to the supernatant to generate the second precipitate.

23. The method of claim 11, wherein about one volume of ethanol is added to the supernatant to generate the second precipitate.
